(19) United States
(12) Patent Application Publication
(10) Pub. No.: US 2017/0258611 A1
(43) Pub. Date: Sep. 14, 2017

(54) DEVICES AND METHODS FOR FORMING STENTS IN VIVO

(71) Applicant: Mayo Foundation for Medical Education and Research, Rochester, MN (US)

(72) Inventors: Brandon J. Tefft, Rochester, MN (US); Gurpreet S. Sandhu, Rochester, MN (US); Dan Dragomir-Daescu, Rochester, MN (US); Susheil Uthamaraj, Rochester, MN (US)

(73) Assignee: Mayo Foundation for Medical Education and Research, Rochester, MN (US)

(21) Appl. No.: 15/309,635

(22) PCT Filed: May 7, 2015
(86) PCT No.: PCT/US15/29713

§ 371 Date: Nov. 8, 2016

Related U.S. Application Data
(60) Provisional application No. 61/991,007, filed on May 9, 2014.

(51) Int. Cl. A61F 2/945; A61L 31/00; A61L 31/14

(52) U.S. Cl. CPC A61F 2/945; A61L 31/145; A61L 31/00; A61L 31/148; A61F 2/958

(57) ABSTRACT

Materials and methods for generating conformable stents at sites of stenosis in bodily vessels (e.g., blood vessels) are provided herein.

### CROSS-REFERENCE TO RELATED APPLICATIONS

This application claims benefit of priority from U.S. Provisional Application Ser. No. 61/991,007, filed on May 9, 2014.

### TECHNICAL FIELD

This document relates to materials and methods for treating sites of stenosis in bodily vessels (e.g., blood vessels).

### BACKGROUND

Angioplasty is the mechanical widening of a narrowed blood vessel or a blood vessel that has become obstructed due to, for example, atherosclerosis. The term “angioplasty” has come to include all manner of vascular interventions typically performed in a minimally-invasive or “percutaneous” method.

Coronary angioplasty, also referred to as percutaneous coronary intervention, is a therapeutic procedure to treat stenotic (narrowed) coronary arteries of the heart found in coronary heart disease. Stenotic segments can result from the build-up of cholesterol-laden plaques that form due to atherosclerosis, for example. Coronary angioplasty typically is performed by an interventional cardiologist.

Peripheral angioplasty refers to the use of mechanical widening to open blood vessels other than the coronary arteries. This procedure often is referred to as percutaneous transluminal angioplasty (PTA), and is most commonly performed to treat narrowing in the leg arteries, especially the common iliac, external iliac, superficial femoral, and popliteal arteries. PTA also can be used to treat narrowing of veins. In addition, atherosclerotic obstruction of the renal artery and carotid artery stenosis can be treated with angioplasty.

Any of these angioplasty procedures can include placement of a stent to prevent or counteract constriction of localized blood flow. The stent can act as a scaffold, remaining in place permanently to help keep the vessel open. A stent typically is inserted through a main artery in the groin (femoral artery) or arm (brachial artery) on a wire or catheter (e.g., a balloon catheter, in the case of balloon angioplasty), and threaded up to the narrowed section of the vessel. In a balloon angioplasty procedure, the balloon can be inflated to push the plaque out of the way and expand the vessel. In a balloon angioplasty/stent placement procedure, a stent either can be stretched open by the balloon at the same time as the artery, or can be inserted into the vessel immediately after the angioplasty procedure. Once in place, the stent helps to hold the vessel open, thus improving blood flow. In addition, since angioplasty can result in tears or dissections in the intimal lining of blood vessels, stents can push back these flaps of tissue and thereby maintain vessel patency.

### SUMMARY

This document is based in part on the development of stents that can be pre-packaged in an uncured, fluid form into a desired configuration within a wrapper, combined with a placement device, inserted into a vessel and positioned at a site of stenosis, and cured to form a solid, rigid structure.

Thus, this document provides materials and methods for making and placing stents in narrowed or obstructed vessels (e.g., blood vessels) at the site of stenosis. The stents made by the methods provided herein can conform to irregularly shaped areas within vessels at the site of narrowing or obstruction. Such stents can be particularly useful, for example, at sites where a vessel is of non-uniform diameter.

In one aspect, this document features a method for making a stent. The method can include (a) providing a pre-packaged, fluid stent material, where the pre-packaged stent material is contained within a wrapper and positioned on the outer surface of a delivery device, (b) inserting the delivery device into a vessel, and advancing the pre-packaged stent material to a site of stenosis within the vessel, (c) exposing the stent material to a curing agent such that the stent material solidifies and/or polymerizes, and (d) removing the delivery device from the vessel. The delivery device can include an angioplasty balloon, where the pre-packaged stent material is positioned on the outer surface of the balloon. The method can further include inflating the balloon to move the stent material toward the inner surface of the vessel. The stent material can be a pre-polymer. The stent material can be selected from the group consisting of hydrogels, homopolymers, and copolymers. The stent material can be an acrylic compound. The wrapper can include a biodegradable material [e.g., one or more of poly(glycerol sebacate), polycaprolactone, cellulose, and poly(lactic-co-glycolic acid)]. The curing agent can be UV light.

In another aspect, this document features an article of manufacture that includes a pre-packaged, fluid stent material contained within a wrapper and positioned on the outer surface of a delivery device, wherein the stent material solidifies and/or polymerizes after exposure to a curing agent. The delivery device can include an angioplasty balloon, and the pre-packaged, fluid stent material can be positioned on the outer surface of the balloon. The stent material can be a pre-polymer. The stent material can be selected from the group consisting of hydrogels, homopolymers, and copolymers. The stent material can be an acrylic material. The wrapper can include a biodegradable material (e.g., one or more of poly(glycerol sebacate), polycaprolactone, cellulose, and poly(lactic-co-glycolic acid)). The curing agent can be UV light

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1A is a cross-sectional drawing of a blood vessel having a plaque therein. FIG. 1B is a cross-sectional drawing of the blood vessel after placement of a stent.

FIGS. 2A and 2B show a CAD rendering of one embodiment of a liquid pre-polymer filled wrapper in flat (2A) and wrapped (2B) configurations.

FIGS. 3A and 3B are pictures of one embodiment of a stent wrapped around a balloon.

FIG. 4 is a CAD rendering showing an embodiment of a wrapped stent constrained by a sleeve on a delivery device.

Like reference symbols in the various drawings indicate like elements.

### DETAILED DESCRIPTION

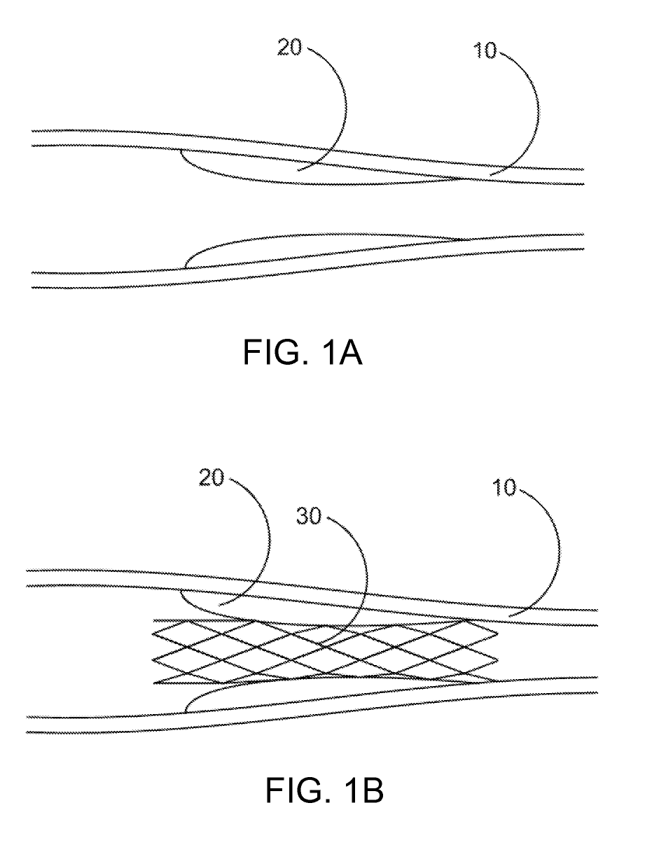

This document provides conformable stents, as well as materials and methods for making such stents. A conformable stent can be generated by, for example, placing a fluid substance (e.g., a liquid or gelatinous substance) at the site of a narrowing or obstruction in a vessel and allowing or causing the fluid to cure, resulting in a rigid structure that can maintain the vessel in an open state. As shown in FIG. 1A, for example, vessel 10 can have an area of stenosis caused by plaque 20. Stent 30, depicted in FIG. 1B, can be placed at the site of plaque 20 to hold vessel 10 in an open state, thus facilitating blood flow.

In general, a stent as provided herein can be made from a fluid (e.g., a liquid or gel pre-polymer) material contained within a wrapping that provides a particular shape or configuration for the stent material. The wrapping containing the stent material can be placed on a delivery device, which then can be deployed into a vessel to a site of stenosis. After delivery to the desired site, the stent material can be allowed or caused to solidify (e.g., through curing or setting), and the delivery device can be removed to leave the “conformed” stent in place, thus holding the stenotic portion of the vessel in an open state.

Any suitable material can be used to form a stent as described herein. Substances that can be useful in the stents provided herein can have one or more of the following characteristics: they can be inert with regard to bodily fluids/materials, can cure quickly (e.g., in two minutes or less, 90 seconds or less, or one minute or less), can avoid emboli, can be non-thrombogenic, can be solid at body temperature, can be liquid or gelatinous at either higher than body or lower than body temperature, can be made of a dielectric or electrically conducting structure that is compatible with common antiplatelet and other drugs, can be non-toxic/biocompatible, and can biodegrade over a period of weeks. Biocompatible polymers that can be used to generate stents as described herein include, for example, the polymers disclosed in U.S. Pat. No. 6,281,263, which is incorporated herein by reference in its entirety. In some embodiments, the stent material can have properties that allow for reversal of its physical state (e.g., properties that allow the material of a solidified stent to return to a liquid state).

Suitable biocompatible polymers and pre-polymers include, for example, hydrogels, copolymers, and homopolymers. For example, modified cellulose and cellulosic derivatives (e.g., cellulose acetate) can be useful. A “modified cellulosic derivative” is a cellulosic polymer that is surface modified by covalently linking pendant biocompatible surface groups to the cellulosic substrate polymer, rendering it more biocompatible. Such surface groups can include those known in the art, such as, e.g., albumin.

The term “homopolymers” includes materials that also can be identified as slightly cross-linked homopolymers (i.e., they contain a relatively small amount of a second component either intrinsic in the production of the monomer or added purposely to insure enough cross-linking so as to protect the homopolymer from slowly dissolving away in an aqueous media such as blood). An example of such a homopolymer is hydroxyethyl methacrylate (HEMA).

With regard to hydrogels, suitable polymers either can be regular homopolymers containing substantially no other material in their matrices, or they can be copolymers prepared from two or more monomers such as, for example, styrene and vinyl acetate. In some cases, copolymer tailoring with various monomers can enhance the desirable properties of the biocompatible substance. Examples of monomers that can be copolymerized include, without limitation, hydroxyethyl methacrylate and glycidyl methacrylate.

A particular example of a useful liquid pre-polymer is LOCTITE® 3922™ (Henkel Corp., Rocky Hill, Conn.). This acrylic compound is capable of curing quickly (after about 20 seconds of exposure to UV light), and is ISO-10993 Biological Tested for medical device use. LOCTITE® 3922™ also fluoresces under UV light, has a low viscosity (15-450 cP), has a large depth of cure, and has a large range of power densities for curing. Other potentially useful LOCTITE® compounds include LOCTITE® 3301™ (acrylic; viscosity 90-210 cP), LOCTITE® 4306™ (cyanoacrylate; viscosity 10-35 cP), LOCTITE® 4310™ (cyanoacrylate, viscosity 100-250 cP), LOCTITE® 3311™ (acrylic, viscosity 200-400 cP), LOCTITE® 4311™ (cyanoacrylate, viscosity 600-1500 cP), LOCTITE® 3971™ acrylic, viscosity 200-425 cP), LOCTITE® 5055™ (silicone, viscosity 200-850 cP), LOCTITE® 3922™ (acrylic, viscosity 15-450 cP), and LOCTITE® 3921™ (acrylic, viscosity 80-220 cP).

Useful pre-polymer materials also include powders, liquids, and gels that can swell when cured to enhance mechanical strength while maintaining a small delivery profile.

Terpolymers also can be useful in the stents and methods described herein. Terpolymers are a subclass of copolymers in which three monomers are polymerized. An example of a useful terpolymer is glycidyl methacrylate/N-vinyl pyrrolidone/hydroxyethyl methacrylate (GMA/NVP/HEMA).

Other suitable materials that can be used to generate a stent as described herein include poly(lactic acid), poly(l-lactic acid), poly(glycolic acid), poly(lactic-co-glycolic acid), poly(glycerol sebacate), polyurethane, chitosan, hydroxyapatite, 2-polyhydroxyethyl methacrylate (2-P-HEMA), n-butyl cyanoacrylate (n-BCA), co-extrusions of poly(ε-caprolactone) and poly(ethylene oxide), cellulose acetates (e.g., cellulose diacetate), ethylene vinyl alcohol copolymers, hydrogels (e.g., acrylics), and polyacrylonitrile. In addition, copolymers (prepared with or without various additional monomers) and homopolymers can be polymerized from any of the following monomers: hydroxyalkyl acrylates and hydroxyalkyl methacrylates (e.g., hydroxyethyl acrylate, hydroxypropyl acrylate, and hydroxybutyl methacrylate), epoxy acrylates and epoxy methacrylates (e.g., glycidyl methacrylate), amino alkyl acrylates and amino alkyl methacrylates, N-vinyl compounds (e.g., N-vinyl pyrrolidone, N-vinyl carbazole, N-vinyl acetamide, and N-vinyl succinimide), amino styrenes, polyvinyl alcohols and polyvinyl amines made from suitable polymeric precursors, polyacrylamide and various substituted polyacrylamides, polysaccharides and modified polysaccharides, polyethylene glycol (PEG) and polypropylene glycol (PPG) based polymers, vinyl pyridine, vinyl sulfonate and polyvinyl sulfate, vinylene carbonate, vinyl acetic acid and vinyl crotonic acid, allyl amines and allyl alcohols, and vinyl glycidyl ether and allyl glycidyl ether. Processes and procedures for creating copolymers and/or homopolymers from the above monomers are known in the art. These parameters are not critical to the presently products and methods provided herein, with the caveat that the final copolymer and/or homopolymer is nontoxic for animal (e.g., human) use.

In any of the embodiments provided herein, the substance used to form the stent can contain a radiopaque agent to allow for visualization of the stent during and after deployment. In some embodiments, the stent can contain a therapeutic agent that can be eluted from the stent during and/or after placement.

The stent material can be pre-packaged within a wrapping that provides a particular shape or configuration for the stent, and the stent material and wrapping can be placed on a delivery device. In some embodiments, for example, the delivery device can be a balloon (e.g., an angioplasty balloon). The balloon can be conformable and of relatively low pressure, but may be capable of providing different pressures at different sites. Thus, this document also provides articles of manufacture that can include, for example, a pre-packaged stent material and a delivery device (e.g., a balloon). In some embodiments, an article of manufacture can include a delivery device with a pre-packaged stent material positioned on its outer surface.

A delivery device with a pre-packaged stent material as provided herein can include, for example, a biodegradable wrapper containing the stent material, which can be a fluid (e.g., liquid or gel) pre-polymer, where the wrapper is positioned on (e.g., wrapped around) the outer surface of a support structure such as an elastic balloon (e.g., an angioplasty balloon). Suitable biodegradable materials from which a wrapper can be made include, without limitation, poly(glycerol sebacate), polycaprolactone, cellulose, and poly(lactic-co-glycolic acid). In some embodiments, the wrapper containing the stent material can be held against the support structure by an outer sleeve, which can be removed prior to stent deployment, or which can have a break-away design (e.g., incorporating perforations or adhesives) so that only a portion of the sleeve is retained at the site of stenosis after the stent is placed and cured. Inflation of the balloon can cause the stent material to conform to the native vessel geometry, pushing the uncured stent material toward or against the vessel wall.

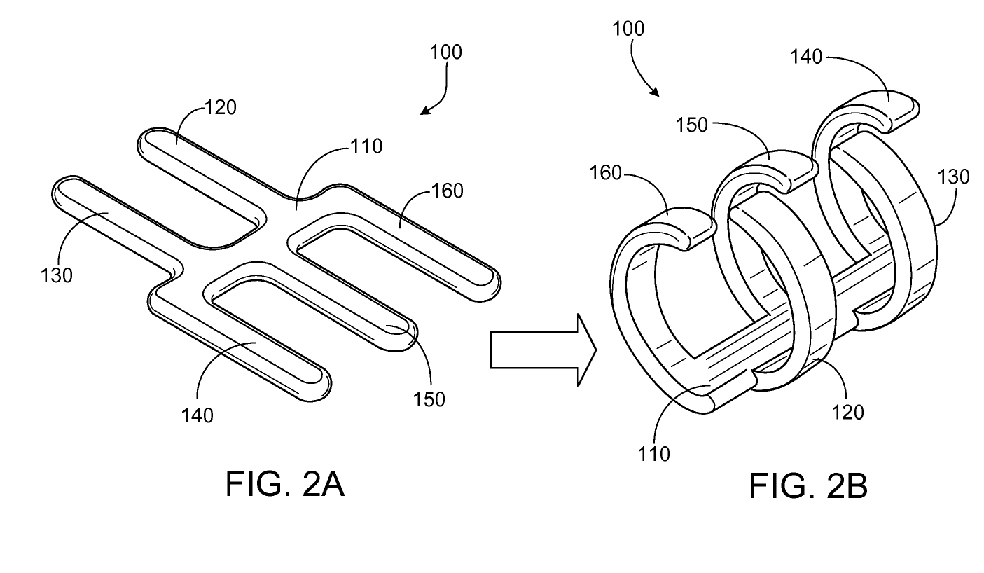

An embodiment of a stent and delivery device as provided herein is depicted in FIGS. 2-4.

FIG. 2A shows an embodiment of a stent 100 in pre-packaged, flat form (e.g., before being positioned on a delivery device). Stent 100 can have an axial portion 110, and lateral extensions 120, 130, 140, 150, and 160. It is to be noted that this is just one exemplary embodiment of a stent as provided herein; a stent can have any suitable number of axial portions (e.g., one, two, three, four, five, six, or more than six axial portions), and any suitable number of lateral extensions (e.g., one, two, three, four, five, six, seven, eight, nine, ten, or more than ten lateral extensions). Further, while the lateral extensions in FIG. 2A are depicted as being perpendicular to the axial portion 110, the extensions can be at any suitable angle to the axial portion(s). The material of stent 100 can be contained within a wrapper (e.g., a biodegradable plastic wrapper, not depicted in FIG. 2A or 2B), and thus can be held in a particular configuration by the wrapper.

FIG. 2B shows the stent embodiment of FIG. 2A after it has been curled around a delivery device (not shown). The lateral extensions 120, 130, 140, 150, and 160 are now curved, such that the stent 100 can be placed inside a blood vessel and cured.

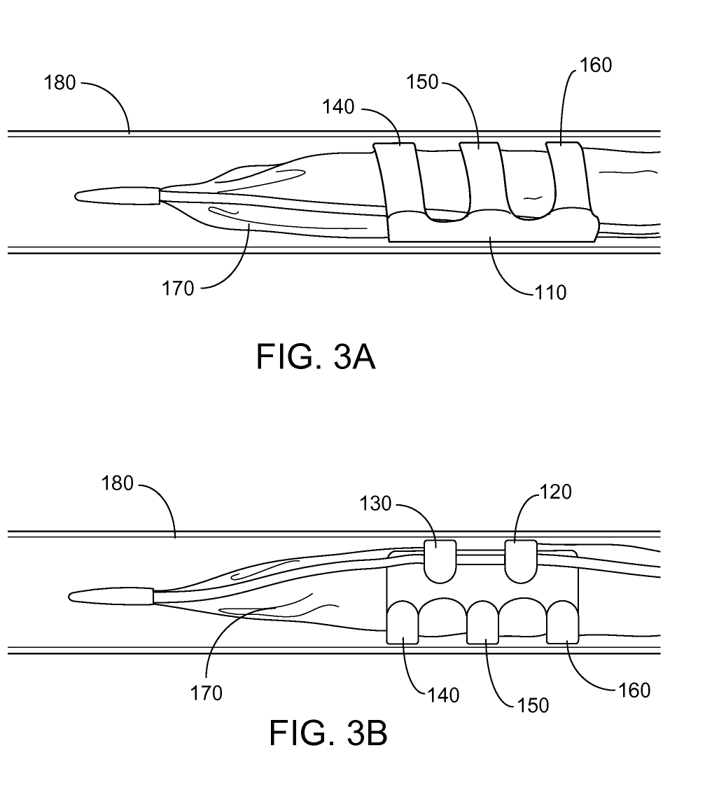

FIGS. 3A and 3B are pictures of a stent as depicted in FIGS. 2A and 2B, curved around an angioplasty balloon and contained within an outer sleeve so that the stent is held against the balloon. FIG. 3A is a side view, showing the axial portion 110 and lateral extensions 140, 150, and 160. FIG. 3B is a top view, showing the lateral extensions 120, 130, 140, 150, and 160. In both FIGS. 3A and 3B, the lateral extensions are held against a balloon 170 by an outer sleeve 180. In use, the stent 100 and balloon 180, contained within the outer sleeve 170, can be positioned at a site of stenosis within a vessel, and the outer sleeve 170 can be removed prior to inflation of the balloon 180 and curing of the stent 100.

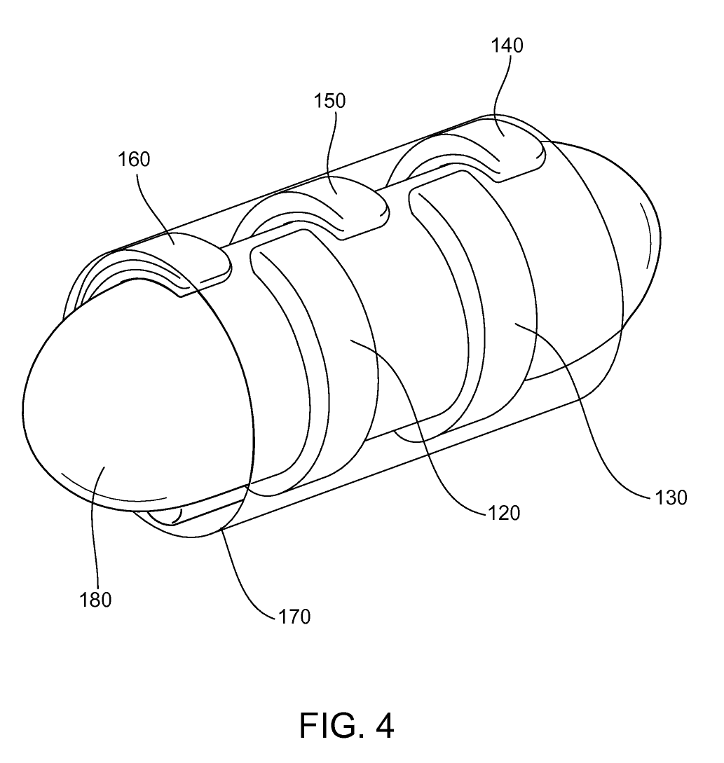

FIG. 4 shows a CAD rendering of a stent as depicted in FIGS. 2A, 2B, 3A, and 3B, in which the lateral extensions 120, 130, 140, 150, and 160 of stent 100 are wrapped around a balloon 180, and contained within an outer sleeve 170.

This document also provides methods for making a stent. The methods can include, for example, providing a pre-packaged stent material (e.g., a pre-polymer in uncured form, packaged in a particular configuration) and a delivery device (e.g., a balloon catheter or an angioplasty balloon), percutaneously delivering the stent material to a stenosed vessel, and causing or allowing the stent material to cure or set. The pre-packaged stent material may or may not be pre-loaded onto the delivery device; in the latter case, the methods can include placing the stent material onto the delivery device. In some embodiments, delivery can be carried out during or after a standard balloon angioplasty procedure to open a vessel lumen, for example.

For example, an angioplasty balloon having a packaged stent material on its outer surface can be inserted into a blood vessel and advanced to a site of stenosis. The balloon can be deployed (e.g., inflated with a fluid) to push the packaged stent material toward the inner wall of the vessel, thus forming a stent that fits precisely into the vessel, including any branch or bifurcation site. In some cases, such stents can be particularly useful in situations where there is significant disparity between the diameter of the vessel proximal and distal to the stenotic segment, for example. The approach described herein can avoid issues such as the need to use differently sized stents at different segments of a vessel.

Once a stent is placed, the stent material can be cured or allowed to set. Any suitable method can be used, including those known in the art. For example, the polymer used in a stent can be cured/polymerized by light/radiation assisted polymerization, by exposure to a particular temperature or range of temperatures (e.g., hot or cold temperatures), by exposure to a particular pH, or by exposure to an enzyme, a catalyst, or a chemical that promotes polymerization.

The delivery device also can provide a means or passage for curing of the stent material. For example, light or radiation can be transmitted through the delivery device to the stent material, thus promoting curing of the material. In some cases, where the stent material is temperature sensitive, a hot or cold substance can be delivered through the device to the location of the stent material to promote curing or setting. In other cases, one or more enzymes, catalysts, or other chemical initiators that act as curing agents can be delivered through the device to the stent material.

Thus, in some embodiments, a curing agent can be injected into the balloon (e.g., to inflate the balloon and solidify or polymerize the stent material). In some cases, this can be accomplished by having perforations and/or secondary tubes within the lumen of the balloon that allow the curing agent to interact with the stent material. The wrapping material also may be permeable or porous to allow for passage of the curing agent, such that the curing agent can contact the stent material. In some embodiments, where the stent material is heat or cold sensitive, the fluid used to inflate the balloon can be hot or cold to activate curing or setting of the stent substance. When the stent material is light sensitive (e.g., UV sensitive), a fiber optic cable can be inserted into the delivery device to transmit UV light to the location of the stent material to promote curing or setting. In some embodiments, an electrode placed within a lumen of the balloon can be used to provide heat, cold, or radiofrequency energy to the stent material, thus promoting curing or setting.

It is to be noted that in some embodiments, a curing agent can be present on the delivery device prior to placement of the stent in a vessel. In such embodiments, for example, the polymer can be like an epoxy, such that when it is deployed, two chemicals are made to mix together to react and harden.

The stents and methods provided herein can also be useful in the complex cardiac venous anatomy to allow, for example, deployment of electrical charge (pacing) to stimulate the myocardium without stimulating extra cardiac structures. For such pacing and other iterations (e.g., branching lesions in an artery), a wire can be positioned within the stent material, or the stent material can be a conductor. The wire may help to hold the stent in place, or may be used as a conductor to be adapted. Thus, a stent can interface via wire to a high energy device (e.g., for ablation and/or defibrillation) placed in the subclavicular region, for example, with the wire (e.g., pacing leads) achieving a near perfect fit onto the myocardial surface from within irregularly shaped and branching second and third order cardiac vein branches. Other benefits with pacing from a conformable stent include that capture of the phrenic nerve can be avoided, and that changes can be made to the resultant pacing vector without the need for multiple leads. In the central nervous system (e.g., venous branches), an additional benefit is that capture from pacing from the distal portion of a conformed stent can be ascertained by a recording electrode “created” on the proximal portion of the injected scaffold/stent.

To prevent extracardiac stimulation, a conformed stent can be placed in two steps. For example, once the balloon has been deployed in, e.g., the coronary venous system, markers can be placed on the balloon or stent packaging to orient the operator as to which side of the stent is facing the myocardial tissue. The stent can be oriented such that the conducting material faces the myocardial surface. In such embodiments, the stent also can contain an insulator that can be oriented to face the extracardiac surface. This can allow for high output stimulation without extracardiac capture.

The materials and methods provided herein also can be used to generate uniquely shaped and sized pacing leads for stimulating devices placed in the central nervous system (e.g., brain and spinal cord), as well as in the vasculature of other organs, including blood vessels draining or supplying muscle, peripheral nerves, dorsal root, and ganglia, where standard placement of a preshaped stent would be difficult.

Stents and wires used for such pacing applications can contain, e.g., titanium, platinum, and other conductors commonly used to create electrodes. In addition, other dielectrics can be used, including those engineered at a monomolecular level to prevent thrombus formation or to allow transmission of direct current in addition to radiofrequency energy.

Further, the stents and methods described herein can be useful for other extravascular applications. For example, a stent provided herein can be deployed in the gastrointestinal tract (e.g., bile duct, bowels, or urinary tubes), and can be used for anastomosis (e.g., in bypass grafts). An electrically conductive stent material in the heart, brain or periphery also may function as a sensor to detect normal or abnormal electrical activity. Such a sensor can be coupled via various means to an “effector” mechanism to generate a neuro-muscular or biologic response at a remote site.

For airway protection applications, a conformable stent can be applied in the trachea and/or bronchus, as well as in the posterior pharynx and nasopharynx. Such methods can be used for treatment of narrowing of the airways and dynamic airway obstruction, such as can occur with sleep apnea and nasopharyngeal obstructions. In such embodiments, a standard stent likely would be difficult to place for various reasons, including the complex geometry of this region. A balloon placed via the nostrils or the pharynx can take the shape of these complex structures, and a conformable stent with gaps left in place for the areas above the soft palates can prevent dynamic compromise of these structures, preventing obstruction. Similarly, tracheal stenosis or bronchial stenosis can be treated with balloon tracheo/bronchoplasty, followed by placement of a conformable stent as described herein.

Conformable stents also can be useful in the biliary tract, the intestinal tract, and the salpingo-uterine system, for example. Like the airway, the biliary tract has a complex, branched anatomy. A stent as provided herein can be used, for example, to treat branch lesions and biliary stenosis, including at second and third order branches of the biliary system. In the intestinal tract, a stent as provided herein also can be used at areas of stenosis that are associated with complex geometry. For example, a stent can be placed at the region of the ileocecal junction, which can be affected in disorders such as ulcerative colitis and Crohn's disease. Placing a stent in such a location via an endoscope typically is difficult because of the disparity in lumen size between the cecum and the ileum. A conformable balloon can take the shape of the respective lumens and allow placement of a conformable stent. In the salpingo-uterine system, a stent as provided herein can be used to treat stenosis associated with infertility, for example. Embodiments that allow elusion of a drug may be particularly useful at such sites.

The conformable stents provided herein also can be used to create varying degrees of penile tumescence. A stent can be created around a balloon and placed in one of the main dorsal or central penile veins. Such a stent may be most similar to the pacing stents described herein. The stent itself, through a conductor, can be attached to a small battery-powered generator that can be placed subcutaneously in anatomic proximity. The material of the stent can be such that the application of a charge will create variation in the sides and physical state of the stent (similar to “curing” with heat, cooling, RF, or DC current, for example). Such a device can be used for treatment of erectile dysfunction by relatively and reversibly occluding the penile vein while opening the arterial vasculature. The generator can be placed subcutaneously, and an external communicating device can be used to control the device.

This document also provides articles of manufacture (kits) containing the conformable stents disclosed herein. An article of manufacture can include, for example, a pre-packaged, fluid stent material that is contained within a wrapper and positioned on the outer surface of a delivery device, where the stent material solidifies and/or polymerizes after exposure to a curing agent (e.g., UV light). In some embodiments, the delivery device can include an angioplasty balloon, such that the pre-packaged, fluid stent material is positioned on the outer surface of the balloon. As described herein, suitable stent materials include, without limitation, pre-polymers, hydrogels, homopolymers, copolymers, and acrylic materials, while suitable wrapper materials include, without limitation, biodegradable material such as poly(glycerol sebacate), polycaprolactone, cellulose, and poly(lactic-co-glycolic acid.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for making a stent, comprising:
a) providing a pre-packaged, fluid stent material, wherein the pre-packaged stent material is contained within a wrapper and positioned on the outer surface of a delivery device;
b) inserting the delivery device into a vessel, and advancing the pre-packaged stent material to a site of stenosis within the vessel;
c) exposing the stent material to a curing agent such that the stent material solidifies and/or polymerizes; and
d) removing the delivery device from the vessel.

2. The method of claim 1, wherein the delivery device comprises an angioplasty balloon, and wherein the pre-packaged stent material is positioned on the outer surface of the balloon.

3. The method of claim 2, further comprising inflating the balloon to move the stent material toward the inner surface of the vessel.

4. The method of claim 1, wherein the stent material is a pre-polymer.

5. The method of claim 1, wherein the stent material is selected from the group consisting of hydrogels, homopolymers, and copolymers.

6. The method of claim 1, wherein the stent material is an acrylic material.

7. The method of claim 1, wherein the wrapper comprises a biodegradable material.

8. The method of claim 7, wherein the wrapper comprises one or more of poly(glycerol sebacate), polycaprolactone, cellulose, and poly(lactic-co-glycolic acid).

9. The method of claim 1, wherein the curing agent is UV light.

10. An article of manufacture comprising a pre-packaged, fluid stent material that is contained within a wrapper and positioned on the outer surface of a delivery device, wherein the stent material solidifies and/or polymerizes after exposure to a curing agent.

11. The article of manufacture of claim 10, wherein the delivery device comprises an angioplasty balloon, and wherein the pre-packaged, fluid stent material is positioned on the outer surface of the balloon.

12. The article of manufacture of claim 10, wherein the stent material is a pre-polymer.

13. The article of manufacture of claim 10, wherein the stent material is selected from the group consisting of hydrogels, homopolymers, and copolymers.

14. The article of manufacture of claim 10, wherein the stent material is an acrylic material.

15. The article of manufacture of claim 10, wherein the wrapper comprises a biodegradable material.

16. The article of manufacture of claim 15, wherein the wrapper comprises one or more of poly(glycerol sebacate), polycaprolactone, cellulose, and poly(lactic-co-glycolic acid).

17. The article of manufacture of claim 10, wherein the curing agent is UV light.
